(19) United States
(12) Patent Application Publication
(10) Pub. No.: US 2022/0204931 A1
(43) Pub. Date: Jun. 30, 2022

(54) BEAD-FREE EX-VIVO EXPANSION OF HUMAN REGULATORY T CELLS

(71) Applicant: The Regents of the University of California, Oakland, CA (US)

(72) Inventors: Qizhi TANG, San Francisco, CA (US); Nikolaos SKARTSIS, San Francisco, CA (US); Flavio VINCENTI, San Francisco, CA (US)

(73) Assignee: The Regents of the University of California, Oakland, CA (US)

(21) Appl. No.: 17/607,354

(22) PCT Filed: Apr. 30, 2020
(86) PCT No.: PCT/US2020/030869

§ 371 Date: Oct. 28, 2021

Related U.S. Application Data
(60) Provisional application No. 62/841,215, filed on Apr. 30, 2019.

(51) Int. Cl. C12N 5/0783; A61K 35/12

(52) U.S. Cl. CPC C12N 5/0637; A61K 35/12; C12N 2501/2301; A61K 2035/124; C12N 2501/2306; C12N 2501/25; C12N 2501/24; C12N 2501/2302

(57) ABSTRACT

The present disclosure relates generally to the manufacture of regulatory T cells (Tregs) for use in adoptive cell therapy. In particular, the present disclosure relates to simplified approaches for the expansion of Tregs ex vivo. Tregs produced in this way are suitable for use in various immunotherapy regimens.

### CROSS-REFERENCE TO RELATED APPLICATIONS

This application claims benefit of U.S. Provisional Application No. 62/841,215, filed Apr. 30, 2019, the disclosure of which is hereby incorporated by reference in its entirety.

### STATEMENT REGARDING FEDERALLY SPONSORED RESEARCH OR DEVELOPMENT

None.

### FIELD

The present disclosure relates generally to the manufacture of regulatory T cells (Tregs) for use in adoptive cell therapy. In particular, the present disclosure relates to simplified approaches for the expansion of Tregs ex vivo. Tregs produced in this way are suitable for use in various immunotherapy regimens.

### BACKGROUND

Regulatory T cells (Tregs) are a small subpopulation of peripheral blood lymphocytes and are critical for controlling tolerance, inflammation, and homeostasis of the immune system. Defects in Tregs have been observed in connection with uncontrolled inflammation and a variety of autoimmune diseases. Accordingly, Tregs are being developed as adoptive cell therapies for treating autoimmune and inflammatory diseases, graft-versus-host disease after bone marrow transplantation, and rejection of solid organ transplants (Bluestone and Tang, Science, 362:154-155, 2018).

Current methods of manufacturing Tregs for preclinical experiments and clinical trials are varied (Ruchs et al., Frontiers in Immunol, 8:1844, 2018). Most methods rely on strong antigenic or mitogenic stimulation of purified Tregs using processes developed for expansion of conventional CD4⁺ T cells and CD8⁺ T cells. In particular, these processes use antibodies to CD3 and CD28 immobilized on beads, artificial antigen presenting cells, or polymeric scaffolds that strongly activate Tregs to drive the cells into proliferation with support of IL-2. Under these unnatural in vitro conditions, Tregs are at risk of losing their identity and function. Thus, there is a need in the art for methods of manufacturing Tregs that result in consistent robust expansion of Tregs without negatively impacting Treg identity and function. Moreover, development of a simplified and adaptable protocol for Treg expansion is desirable to reduce the complexity of cell manufacturing processes and better enable process automation, while maintaining Treg phenotype of the starting cell population.

### BRIEF SUMMARY

The present disclosure relates generally to the manufacture of regulatory T cells (Tregs) for use in adoptive cell therapy. In particular, the present disclosure relates to simplified approaches for the expansion of Tregs ex vivo. Tregs produced in this way are suitable for use in various immunotherapy regimens.

### BRIEF DESCRIPTION OF THE DRAWINGS

FIG. 1 provides a graph depicting the extent of expansion of human Tregs produced using a standard protocol involving anti-CD3 and anti-CD28 monoclonal antibodies conjugated to magnetic beads in comparison to the bead-free protocols of the present disclosure described in Example 1. Abbreviations are as follows: BF1=protocol involving anti-CD28SA Ab and IL-2; BF2=protocol involving anti-CD28SA Ab, IL-2, and IL-6; BF3=protocol involving anti-CD28SA Ab, IL-2, and TNF-alpha; and BF4=protocol involving anti-CD28SA Ab, IL-2, IL-6 and TNF-alpha.

FIG. 2 provides a graph depicting the level of expression of Treg-lineage markers FOXP3, HELIOS and CD27 on human Tregs produced using the bead-free protocols of the present disclosure described in Example 1. Tregs were harvested on day 14. Abbreviations are as described for FIG. 1.

FIG. 3 provides flow cytometry histograms depicting the level of expression of Treg-lineage markers FOXP3, HELIOS, CD62L and CD27 on human Tregs produced using the bead-free protocols of the present disclosure described in Example 1. Tregs were harvested on day 14. Abbreviations are as described for FIG. 1.

FIG. 4 provides flow cytometry histograms depicting the level of expression of Treg-lineage markers HELIOS and CD27 on human Tregs produced using the bead-free protocols of the present disclosure described in Example 1. Tregs were harvested on day 14. Abbreviations are as described for FIG. 1

FIG. 5 provides a graph depicting the extent of expansion of human Tregs produced using a standard protocol involving magnetic beads and anti-CD3 and anti-CD28 monoclonal antibodies in comparison to the BF4 protocol of the present disclosure. Tregs were harvested on day 14.

FIG. 6 provides flow cytometry histograms depicting the level of expression of Treg-lineage markers FOXP3 and HELIOS on human Tregs produced using a standard protocol involving magnetic beads and anti-CD3 and anti-CD28 monoclonal antibodies in comparison to the BF4 protocol of the present disclosure. Tregs were harvested on day 14.

FIG. 7 provides flow cytometry histograms depicting the level of expression of Treg-lineage markers HELIOS and CD27 on human Tregs produced using a standard protocol involving magnetic beads and anti-CD3 and anti-CD28 monoclonal antibodies in comparison to the BF4 protocol of the present disclosure. Tregs were harvested on day 14

FIG. 8A and FIG. 8B provide graphs depicting the level of suppression of pre-activated effector T cell (Teff) and autologous peripheral blood mononuclear cell (PBMC) proliferation respectively, by human Tregs produced using a standard protocol involving magnetic beads and anti-CD3 and anti-CD28 monoclonal antibodies in comparison to the BF4 protocol of the present disclosure.

FIG. 9 provides a graph depicting the level of suppression of effector T cell (Teff) proliferation in the presence and absence of tumor necrosis factor-alpha by human Tregs produced using a standard protocol involving magnetic beads and anti-CD3 and anti-CD28 monoclonal antibodies in comparison to the BF4 protocol of the present disclosure.

FIG. 10 provides a graph depicting the level of expansion of human Tregs produced using two rounds of stimulation with magnetic beads and anti-CD3 and anti-CD28 monoclonal antibodies in the presence of IL-1 (Bead) in comparison to the BF10 protocol of the present disclosure.

### DETAILED DESCRIPTION

The present disclosure relates generally to the manufacture of regulatory T cells (Tregs) for use in adoptive cell therapy. In particular, the present disclosure relates to alternative approaches to the traditional magnetic bead-based or feeder cell-based protocols for the expansion of Tregs ex vivo. Tregs produced in this way are suitable for use in various immunotherapy regimens.

The present disclosure provides methods for production of human regulatory T cells (Tregs), comprising: a) isolating CD4+, CD25+, CD127−/low T cells from a lymphocyte-containing biological sample obtained from a human subject; and b) culturing the T cells in medium comprising a CD28 superagonist (CD28SA) antibody, interleukin-2 (IL-2), and tumor necrosis factor-alpha (TNF-alpha) under conditions effective in producing human Tregs that are CD4+, FOXP3+, HELIOS+, and have a demethylated Treg-specific demethylation region (TSDR). The present disclosure further provides methods for production of human regulatory T cells (Tregs), comprising: a) isolating CD4+, CD25+, CD127−/low T cells from a lymphocyte-containing biological sample obtained from a human subject; and b) culturing the T cells in medium comprising a CD28SA antibody, IL-2) IL-6, and TNF-alpha under conditions effective in producing human Tregs that are CD4+, FOXP3+, HELIOS+, and have a demethylated Treg-specific demethylation region (TSDR). The present disclosure also provides methods for production of human regulatory T cells (Tregs), comprising: a) isolating CD4+, CD25+, CD127−/low T cells from a lymphocyte-containing biological sample obtained from a human subject; and b) culturing the T cells in medium comprising a CD28SA antibody, IL-2, IL-1beta, and TNF-alpha under conditions effective in producing human Tregs that are CD4+, FOXP3+, HELIOS+, and have a demethylated Treg-specific demethylation region (TSDR). In preferred embodiments, the human Tregs are CD3+, CD27+, CD62L+, CD8− and CD19−. Preferred stimulation conditions comprising culturing cells in the presence of IL-6 is referred to as BF4 and BF4a in the examples and figures. A preferred stimulation condition comprising culturing cells in the presence of IL-1beta is referred to as BF10 in the examples and figures.

BF4 and BF10 conditions and variants thereof including culturing T cells in media consisting of the same cytokines, but at different concentrations, are thought to result in the production of a Treg population with advantageous properties as compared to Tregs produced under conditions employing beads or artificial antigen presenting cells to immobilize anti-CD3 and anti-CD28 antibodies. Without being bound by theory, it is thought that immobilization of anti-CD3 and anti-CD28 antibodies is an overly strong, non-physiological stimulus leading to Treg lineage instability and acquisition of pro-inflammatory functions.

As used herein, the terms “CD28 superagonist antibody”, “CD28SA antibody” and “superagonistic anti-CD28 antibody” refer to a CD28-specific monoclonal antibody that is able to activate T-cells in the absence of a T cell receptor activator. Thus in preferred embodiments, step b) does not comprise use of an anti-CD3 antibody and/or does not comprise use of magnetic beads or Fc receptor-expressing feeder cells to cross-link CD28 and CD3 expressed on the surface of the isolated T cells. In some embodiments, the medium further comprises one or both of a tumor necrosis factor receptor 2 agonist (TNFR2a) and interferon-gamma (IFN-gamma). In some embodiments, the TNFR2a is an anti-TNFR2 antibody.

CD28SA monoclonal antibodies have been found to bind to the exposed C″D loop of the immunoglobulin-like domain of CD28, whereas conventional anti-CD28 monoclonal antibodies bind to the exposed F″G loop of CD28, which is critical for B7 binding (Luhder et al., J Exp Med, 197:955-966, 2003). Exemplary CD28SA antibodies suitable for use in the methods of the present disclosure include but are not limited to theralizumab (also known as TAB08, and formerly known as TGN1412) developed by TheraMAB LLC (Moscow, Russia), and ANC28.1 marketed by Ancell Corp (Bayport, Minn.). Amino acid sequences of the variable regions of TGN1412 and variants thereof are described in U.S. Pat. No. 8,709,414.

The bead-free methods of the present disclosure can be used in combination with antigen-specific expansion or selection of Tregs to produce antigen-specific Tregs. For instance, the methods for production of human regulatory T cells (Tregs) may further comprise isolating antigen-specific T cells by staining with a major histocompatibility complex (MHC) class II-peptide multimer and/or culturing the T cells in the presence of a MHC class II-peptide multimer in the presence of IL-2 prior to step b). Methods for antigen-specific expansion employing MHC class II-peptide multimers and methods for adoptive transfer of Tregs are described in U.S. Pat. No. 7,722,862.

Alternatively, the methods for production of human regulatory T cells (Tregs) may further comprise culturing T cells in the presence of allogeneic stimulated B cells (sBc) in the presence of IL-2 prior to step b) and/or during step b). In some embodiments, the T cells comprise a mismatch in HLA-DR in relation to the allogeneic sBc. Methods for antigen-specific expansion employing allogenic sBc and methods for adoptive transfer of Tregs are described in U.S. Pat. No. 9,801,911, the examples of which are incorporated herein by reference.

The methods of the present disclosure may further comprise step c) harvesting the human Tregs, which in some embodiments commences 7 to 18 days after step b) commences. In some embodiments, step c) commences at a minimum of 7, 8, 9, 10, 11, 12, 13, 14, 15, 16 or 17 days after step b) commences and/or at a maximum of 18, 17, 16, 15, 14, 13, 12, 11, 10, 9 or 8 days after step b) commences. The methods of the present disclosure may further comprise step c) harvesting the human Tregs, which in some embodiments commences 11 to 18 days after step b) commences. In some embodiments, step c) commences at a minimum of 11, 12, 13, 14, 15, 16 or 17 days after step b) commences and/or at a maximum of 18, 17, 16, 15, 14, 13, or 12 days after step b) commences. The methods of the present disclosure are suitable for expansion of human Tregs by from about 200 to about 2000 fold. In preferred embodiments, the methods result in the production of at least 200, 600, 1000, 1400, or 1800 fold more human Tregs than were present at the onset of step a). In some embodiments, levels of expression of various markers by the human Tregs are assessed by flow cytometry on the day of harvest. Markers that are assessed may include but are not limited to CD4, CD25, FOXP3, HELIOS, CD27, CD62L, and CD8. Tregs are positive for CD4, CD25, FOXP3, HELIOS, CD27, CD62L and negative for CD8. Also, TSDR demethylation is quantified using bisulfide conversion followed by methylation specific PCR or pyrosequencing. High percentages of TSDR demethylation indicate that the cells produced are a stable lineage of Tregs.

References and claims to methods for treating or preventing a pathological immune response in a human subject in need thereof comprising administering to the subject human Tregs produced using the methods for production of the present disclosure, in their general and specific forms likewise relate to:

a) the use of the human Tregs for the manufacture of a medicament for the treatment or prevention of a pathological immune response; and

b) pharmaceutical compositions comprising the human Tregs for the treatment or prevention of a pathological immune response.

As used herein, the term “pathological immune response” encompasses autoimmune diseases, autoinflammatory diseases, allograft rejection, and graft versus host disease. “Autoimmune diseases” involve immune recognition resulting in direct damage to self-tissue and functional impairments. Pathologically, autoimmune diseases are typically driven by cells of the adaptive immune system. Autoimmune diseases include but are not limited to rheumatoid arthritis, multiple sclerosis, systemic lupus erythematosus, pemphigus, psoriasis, type I diabetes, celiac disease, and Sjogren's syndrome. “Autoinflammatory diseases” involve spontaneous activation, or over-reaction of the immune system to non-self-antigens (e.g., environmental, food, commensal or other antigens) resulting in indirect (bystander) damage to self-tissue and functional impairments. Pathologically, autoinflammatory diseases are typically dominated by cells of the innate immune system. Examples of autoinflammatory diseases include but are not limited to inflammatory bowel disease, amyotrophic lateral sclerosis and other neurodegenerative diseases, allergic airway disease, and chronic obstructive pulmonary disease.

The present disclosure further provides pharmaceutical compositions comprising the human Tregs and a physiologically acceptable buffer such as saline or phosphate-buffered saline. An effective amount of the pharmaceutical composition for adoptive cell therapy comprises from 10⁷ to 10¹¹ (10 million to 100 billion) of the human Tregs (see, e.g., Tang and Lee, Curr Opin Organ Transplant, 17:349-354, 2012). In some instances, the human Tregs are administered either locally to the diseased tissue (e.g., by intra-articular infusion to affected joints when treating rheumatoid arthritis), or systemically (e.g., by intravenous infusion when treating systemic lupus erythematosus). In some embodiments, the Tregs are administered either as a single infusion, or as multiple infusions for better engraftment and prolonged effects. Local infusion may comprise administration of from 10⁷ to 10⁹, whereas systemic infusion may comprise administration of 10⁹ to 10¹¹ Tregs. Treatment or prevention of solid organ transplantation may comprise administration of 10⁹ to 10¹¹ Tregs, while treatment or prevention of graft-versus-host disease may comprise administration of 10¹⁰ to 10¹¹ Tregs.

As used herein and in the appended claims, the singular form “a,” “an” and “the” includes plural forms unless indicated otherwise. For instance, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 200 fold refers to 180 fold to 220 fold and includes 200 fold).

An “effective amount” of an agent disclosed herein is an amount sufficient to carry out a specifically stated purpose. An “effective amount” may be determined empirically in relation to the stated purpose. An “effective amount” or an “amount sufficient” of an agent is that amount adequate to affect a desired biological effect, such as a beneficial result, including a beneficial clinical result. The term “therapeutically effective amount” refers to an amount of an agent (e.g., human Tregs) effective to “treat” a disease or disorder in a subject (e.g., a mammal such as a human). An “effective amount” or an “amount sufficient” of an agent may be administered in one or more doses.

The terms “treating” or “treatment” of a disease refer to executing a protocol, which may include administering one or more drugs to an individual (human or otherwise), in an effort to alleviate a sign or symptom of the disease. Thus, “treating” or “treatment” does not require complete alleviation of signs or symptoms, does not require a cure, and specifically includes protocols that have only a palliative effect on the individual. As used herein, and as well-understood in the art, “treatment” is an approach for obtaining beneficial or desired results, including clinical results. Beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission. “Treatment” can also mean prolonging survival of a recipient of an allograft as compared to expected survival of a recipient of an allograft not receiving treatment. “Palliating” a disease or disorder means that the extent and/or undesirable clinical manifestations of the disease or disorder are lessened and/or time course of progression of the disease or disorder is slowed, as compared to the expected untreated outcome.

### ENUMERATED EMBODIMENTS

In the embodiments described below, any reference to embodiment 1, encompasses one or both of embodiment 1A and embodiment 1B.

1A. A method for the production of human regulatory T cells (Tregs), comprising:

a) isolating CD4+, CD25+, CD127−/low T cells from a lymphocyte-containing biological sample obtained from a human subject; and

b) culturing the T cells in medium comprising a CD28 superagonist (CD28SA) antibody, interleukin-2 (IL-2), interleukin-6 (IL-6), and tumor necrosis factor-alpha (TNF-alpha) under conditions effective in producing human Tregs that are CD4+, FOXP3+, HELIOS+, and have a demethylated Treg-specific demethylation region (TSDR), optionally wherein the human Tregs are CD62L+, and TNFR2+.

1B. A method for the production of human regulatory T cells (Tregs), comprising:

a) isolating CD4+, CD25+, CD127−/low T cells from a lymphocyte-containing biological sample obtained from a human subject; and

b) culturing the T cells in medium comprising a CD28 superagonist (CD28SA) antibody, interleukin-2 (IL-2), and tumor necrosis factor-alpha (TNF-alpha) under conditions effective in producing human Tregs that are CD4+, FOXP3+, HELIOS+, and have a demethylated Treg-specific demethylation region (TSDR), optionally wherein the human Tregs are CD62L+, and TNFR2+.

2. The method of embodiment 1, wherein step b) does not comprise use of an anti-CD3 antibody.

3. The method of embodiment 1 or embodiment 2, wherein step b) does not comprise use of magnetic beads or Fc receptor-expressing feeder cells to cross-link CD28 and CD3 of the isolated T cells.

4. The method of any one of embodiments 1-3, wherein the medium further comprises one or both of a tumor necrosis factor receptor 2 agonist (TNFR2a) and interferon-gamma (IFN-gamma); optionally wherein the TNFR2a is an anti-TNFR2 antibody.

5. The method of any one of embodiments 1B-4, wherein the medium further comprises one or both of IL-6 and IL-1beta, optionally wherein the medium further comprises IL-1beta but not IL-6, optionally wherein the medium further comprises IL-6 IL-1beta but not IL-1beta.

6. The method of any one of embodiments 1-5, wherein the lymphocyte-containing biological sample is selected from the group consisting of whole blood, a leukapheresis product, and peripheral blood mononuclear cells (PBMC); optionally wherein the biological sample is either fresh or cryopreserved after being obtained from the human subject and subsequently thawed prior to step a).

7. The method of any one of embodiments 1-6, wherein the CD4+, CD25+, CD127−/low T cells of step a) are isolated from the biological sample by fluorescence-activated cell sorting (FACS) or magnetic-activated cell sorting (MACS).

8. The method of any one of embodiments 1-7, further comprising step c) harvesting the human Tregs.

9. The method of embodiment 8, wherein step c) commences 7-18 days after step b) commence, optionally wherein step c) commences 11-18 days after step b) commences.

10. The method of embodiment 9, wherein the human Tregs comprise from about 200 to about 2000 fold more cells than the CD4+, CD25+, CD127−/low T cells at the onset of step a).

11. A pharmaceutical composition comprising from 10⁷ to 10¹¹ of the human Tregs produced using the method of any one of embodiments 1-10, and a physiologically acceptable buffer.

12. A method for treating or preventing a pathological immune response in a human subject in need thereof, the method comprising: administering to the human subject an effective amount of the pharmaceutical composition of embodiment 11; optionally wherein the effective amount of the pharmaceutical composition comprises from 10⁷ to 10¹¹ of the human Tregs and is infused intravenously over a 20-40 minute interval to the human subject.

13. The method of embodiment 12, wherein the pathological immune response is an autoimmune or autoinflammatory disease.

14. The method of embodiment 13, wherein the autoimmune or autoinflammatory disease is selected from the group consisting of rheumatoid arthritis, multiple sclerosis, amyotrophic lateral sclerosis, systemic lupus erythematosus, pemphigus, psoriasis, type I diabetes, celiac disease, and inflammatory bowel disease; optionally wherein the autoimmune or autoinflammatory disease is an inflammatory bowel disease selected from the group consisting of ulcerative colitis, and Crohn's disease.

15. The method of embodiment 13 or 14, wherein the method is effective in reducing a symptom, or is effective in inhibiting progression of the autoimmune or autoinflammatory disease; optionally wherein inhibiting progression of the autoimmune or autoinflammatory disease comprises inhibiting tissue destruction.

16. The method of embodiment 12, wherein the pathological immune response is rejection of a hematopoietic allograft or a solid organ allograft.

17. The method of embodiment 16, wherein the pathological immune response is rejection of a hematopoietic allograft, and the hematopoietic allograft is a bone marrow graft or a peripheral blood stem cell graft.

18. The method of embodiment 16, wherein the pathological immune response is rejection of a solid organ allograft, and the solid organ allograft is selected from the group consisting of cardiac, lung, cardiac/lung, kidney, pancreas, kidney/pancreas, liver, intestine, pancreatic islet, and skin allografts.

19. The method of embodiment 16, wherein the method is effective in reducing a symptom of acute and/or chronic rejection, or is effective in prolonging survival of the organ allograft.

20. The method of embodiment 12, wherein the pathological immune response is a graft versus host disease (GvHD).

21. The method of embodiment 20, wherein the method is effective in reducing a symptom of acute and/or chronic GvHD, or is effective in inhibiting damage to skin, liver, lung, and/or gut of the host.

22. The method of embodiment 12, wherein the method is effective in increasing Treg percentages over baseline in the human subject.

23. A method for inhibiting proliferation of human effector T cells (Teffs), the method comprising: contacting human CD4+, CD25−, CD127+ Teffs with the human Tregs produced using the method of any one of embodiments 1-10 under conditions effective in inhibiting proliferation of the Teffs; optionally wherein the contacting is done in the presence of TNF-alpha.

24. The method or composition of any one of embodiments 1-23, wherein the method for production of the human Tregs is good manufacturing practice (GMP)-compliant.

### EXAMPLES

The present disclosure is described in further detail in the following examples, which are not in any way intended to limit the scope of the disclosure as claimed. The attached figures are meant to be considered as integral parts of the specification and description of the disclosure. The following examples are offered to illustrate, but not to limit the claimed disclosure.

In the experimental disclosure which follows, the following abbreviations apply: Ab (antibody); allo (allogeneic); BF (bead free); CD28 superagonist (CD28SA); FACS (fluorescence-activated cell sorting); IL-1β (interleukin-1beta); IL-2 (interleukin-2); IL-6 (interleukin-6); IFNγ (interferon-gamma); PBMC (peripheral blood mononuclear cell); Teff (effector T cell); TNFα (tumor necrosis factor-alpha); TNF receptor II agonist antibody (TNFR2a); Treg (regulatory T cell); TSDR (Treg-specific demethylation region); and UCSF (University of California San Francisco).

### Example 1

### Development of a Bead-Free Method of Producing Regulatory T Cells (Tregs)

This example describes development of a bead-free method of expanding human Tregs ex vivo.

Treg Isolation.

Human peripheral mononuclear cells were isolated from peripheral blood samples using a ficoll gradient before being washed twice and stained with antibodies against CD4 (anti-CD4 PerCP, clone SK3, BD Biosciences, Catalog No. 347324), CD25 (anti-CD25 APC, clone 2A3, BD Biosciences, Catalog No. 340939) and CD127 (anti-CD127 PE, clone HIL-7R-M21, BD Biosciences, Catalog No. 557938). CD4+CD25highCD127−/low Tregs were isolated by fluorescence-activated cell sorting (FACS).

Ex-Vivo Treg Expansion.

1×10⁵ CD4+CD25+CD127−/low Tregs were plated in single wells of 48-well plates in 500 ml of T cell media (RPMI containing 5% FBS, penicillin/streptomycin, HEPES, sodium pyruvate, glutamax and non-essential amino acids). Alternatively, X-VIVO15 containing human AB serum is used. T cells were stimulation with either 1-10 μg/mL of a CD28SA Ab (ANC28.1, clone 5D10, Ancell Corp., Catalog No. 177-020) or magnetizable polymer beads covalently coupled to anti-CD3 and anti-CD28 antibodies (anti-CD3/CD28 beads) at 1:1 bead to cell ratio. The anti-CD3/CD28 beads were Dynabeads™ Human T-Activator CD3/CD28 for T Cell Expansion and Activation (ThermoFisher Scientific, Catalog No. 111.31D). The Bead-Free (BF) conditions tested are shown in Table 1-1. Cells were supplemented with fresh media on days 2, 5, 7, 9, 11 and 13. Human recombinant IL-2 was supplemented at 300 IU/mL on days 0, 2, 5, 7, 9, 11 and 13. Human recombinant IL-6 (Peprotech, Catalog No. 200-06) was supplemented at 15, 50 and 150 ng/mL on days 0, 2 and 5. Human recombinant TNFα (Peprotech, Catalog No. 300-01A) was supplemented at 50 ng/mL on days 0, 2 and 5. TNFR2a (clone MR2-1, HycultBiotech, Catalog No. HM2007-FS) was supplemented at 2.5 μg/mL on days 0, 2 and 5. Human recombinant IFNγ (Peprotech, Catalog No. 300-02) was supplemented at 40 ng/mL on days 0, 2 and 5. Human recombinant IL-1β (Peprotech, Catalog No. 200-01B) was supplemented at 50 ng/mL on days 0, 2 and 5. Cells were counted on days 5, 7, 9, 11, 13 and 14, and harvested on day 14 for analysis.

TABLE 1-1
Bead-Free Treg Stimulation Conditions

| | CD28SA Ab | IL-2 | IL-6 | TNFα | TNFR2a | IFNγ | IL-1β |
|---|---|---|---|---|---|---|---|
| Condition | (μg/ml) | (IU/ml) | (ng/ml) | (ng/ml) | (μg/ml) | (ng/ml) | (ng/ml) |
| BF1 | 4 | 300 | — | — | — | — | — |
| BF1a | 2 | 300 | — | — | — | — | — |
| BF2 | 4 | 300 | 150 | — | — | — | — |
| BF3 | 4 | 300 | — | 50 | — | — | — |
| BF4 | 4 | 300 | 150 | 50 | — | — | — |
| BF4a | 4 | 300 | 15 | 50 | — | — | — |
| BF4b | 4 | 300 | 50 | 50 | — | — | — |
| BF5 | 4 | 300 | — | — | 2.5 | — | — |
| BF6 | 4 | 300 | 150 | — | 2.5 | — | — |
| BF7 | 4 | 300 | 150 | 50 | 2.5 | — | — |
| BF8 | 4 | 300 | — | — | — | 40 | — |
| BF9 | 4 | 300 | 50 | — | — | 40 | — |
| BF10 | 5 | 300 | — | 50 | — | — | 50 |

Flow Cytometry.

Samples containing 1×10⁵ ex-vivo expanded Tregs were harvested on day 14 of culture and stained with antibodies against CD4, CD27, FOXP3, and HELIOS for immunophenotyping.

Treg-Specific Demethylation Region (TSDR) Analysis.

Samples containing 5×10⁵ ex-vivo expanded Tregs were harvested on day 14 of culture and methylation of the FOXP3 gene locus was assessed by pyrosequencing.

In-Vitro Suppression Assays.

Ex-vivo expanded Tregs cultured under different conditions (as described above) were harvested and washed twice prior to being co-cultured with either pre-activated Teff or autologous PBMC. CD4+CD25lowCD127+ T cells isolated from PBMC by FACS were stimulated with anti-CD3/CD28 beads at 1:1 cell to bead ratio. Fresh cell culture media was added on days 2, 5, 7, 9, 11, 13 and 15 (or 2, 5, and 7) to obtain a pre-activated Teff population. PBMC were cryopreserved and thawed before use. In vitro suppression assays were setup with 50,000 pre-activated Teff or PBMC and various ratios of Tregs. In some assays, 50 ng/ml TNFα was added to co-culture wells. Tritiated-thymidine was added on day 4 of co-culture for the last 16-18 hours, and cell proliferation was determined by measurement of tritiated-thymidine incorporation.

### Results

BF1 and BF1a conditions were compared with a standard anti-CD3/CD28 bead condition, in the presence or absence of IL-2. Treg expansion by stimulation with a CD28 superagonist (CD28SA) antibody was found to be dependent on the concentration of CD28SA Ab and the presence of IL-2. In brief, greater expansion of Tregs was observed when 4 μg/ml rather than 2 μg/ml CD28SA Ab was present. Additionally, both BF1 and BF1a conditions resulted in greater and prolonged expansion of Tregs than did the standard anti-CD3/CD28 bead condition. Microscopic images taken on day 5 of the culture showed strong activation of Tregs by CD28SA Ab in the presence of IL-2, and complete absence of activation-associated cell clustering in the absence of IL-2. In contrast, anti-CD3/CD28 beads activated Tregs in both the presence and absence of IL-2.

Three different populations of T cells were isolated by FACS and stimulated under BF1 conditions or a standard anti-CD3/CD28 bead condition for seven days. Microscopic images taken on day 7 of the culture showed that CD28SA Ab preferentially activates CD4+CD25+CD127−/low Tregs, over CD4+CD25−CD127high T effector cells (Teff) and CD8+ T cells. Preferential activation of Tregs was not observed when anti-CD3/CD28 beads were employed.

BF1 and BF2 conditions were compared with a standard anti-CD3/CD28 bead condition. The ex vivo expansion rate of CD28SA Ab-stimulated Tregs was not found to be significantly affected by the addition of IL-6 in the culture and rates of both BF1 and BF2 were superior to that observed with bead stimulation.

BF1 and BF3 conditions were compared with a standard anti-CD3/CD28 bead condition. The ex vivo expansion rate of CD28SA Ab-stimulated Tregs was not found to be significantly affected by the addition of TNFα in the culture and rates of both BF1 and BF3 were superior to that observed with bead stimulation.

BF1 and BF4 conditions were compared with a standard anti-CD3/CD28 bead condition. The ex vivo expansion rate of CD28SA Ab-stimulated Tregs was improved by the addition of IL-6 and TNFα in the culture. Microscopic images of bead-stimulated Tregs and BF4-stimulated Tregs on day 5 of culture showed extensive cell clustering in the BF4 condition indicative of strong Treg activation and proliferation. Additionally, ex-vivo expansion of CD28SA Ab-stimulated Tregs exposed to IL-6 and TNFα was found to be prolonged and robust. This is advantageous as it obviates the need for Treg re-stimulation, which in turn risks destabilization of Tregs.

BF4, BF4a and BF4b conditions were compared with a standard anti-CD3/CD28 bead condition. IL-6 was found to enhance Treg expansion under a broad range of concentrations (15, 50 or 150 ng/ml) from cells isolated from the peripheral blood of three different human donors (50 year old female, 21 year old male, and 33 year old male).

BF1 and BF6 conditions were compared with a standard anti-CD3/CD28 bead condition. The ex vivo expansion rate of CD28SA Ab-stimulated Tregs was improved by the addition of IL-6 and TNFR2a in the culture.

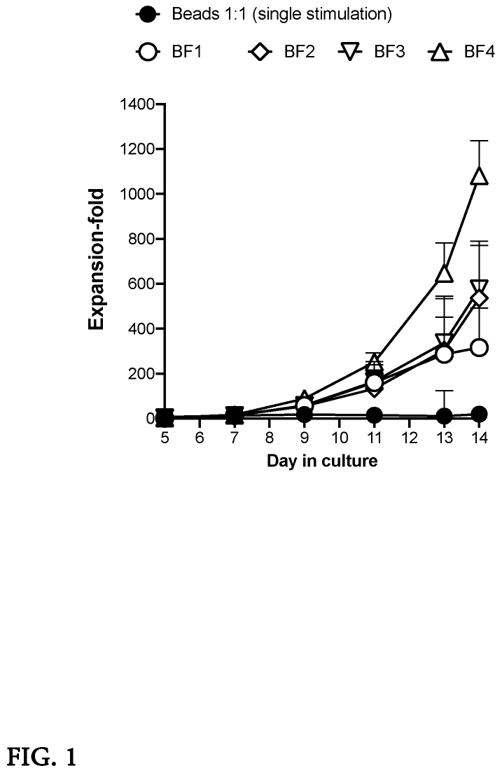

A comparison of ex vivo expansion of Tregs under BF1, BF2, BF3, BF4, and a standard anti-CD3/CD28 bead condition is shown in FIG. 1. A more extensive comparison of overall ex vivo expansion of Tregs after 14 days of culture is shown in Table 1-2.

TABLE 1-2
Ex Vivo Expansion Efficacy

| Stimulation | Fold Expansion | ~Range (minimum |
|---|---|---|
| Condition | ± SEM | to maximum) |
| Beads 1:1 (1 stimulation) | 37.4 ± 26.7 | 7 to 70 |
| Beads 1:1 (2 stimulations) | 415.8 ± 572.3 | 40 to 1560 |
| BF1 | 305.5 ± 137.3 | 46 to 460 |
| BF2 | 536.5 ± 223.9 | 218 to 860 |
| BF3 | 577.3 ± 202.5 | 330 to 880 |
| BF4 | 935.3 ± 431.4 | 365 to 1560 |
| BF4a | 1054 ± 567.4 | 368 to 1540 |
| BF4b | 834.1 ± 365.9 | 352 to 1200 |
| BF5 | 525.0 ± 0 | 525 |
| BF6 | 1100 ± 141.4 | 1000 to 1200 |
| BF7 | 1125 ± 75 | 1050 to 1200 |
| BF8 | 530.0 ± 400 | 130 to 930 |
| BF9 | 770.0 ± 430 | 340 to 1200 |
| BF10 | 742.5 ± 0 | 743 |

BF8 and BF9 conditions were compared with a standard anti-CD3/CD28 bead condition. The ex vivo expansion rate of CD28SA Ab-stimulated Tregs was improved by the addition of one or both of IL-6 and IFNγ in the culture.

BF10 condition was compared with a standard anti-CD3/CD28 bead condition. The ex vivo expansion rate of CD28SA Ab-stimulated Tregs was improved by the addition of both TNFα and IL-1β in the culture. Additionally, 62% of the Treg population produced under the BF10 condition are TNFR2+, CD25+ versus 47% of the Treg population produced under the BF1 condition in the presence of CD28SA Ab and IL-2 and absence of TNFα and IL-1β. Interestingly, Tregs produced under the BF10 condition expressed higher levels of CD71 than did Tregs produced under the BF1 condition. CD71 is the transferrin receptor, which is upregulated in activated T cells and indicative of cells that have entered an anabolic state, conducive for proliferation.

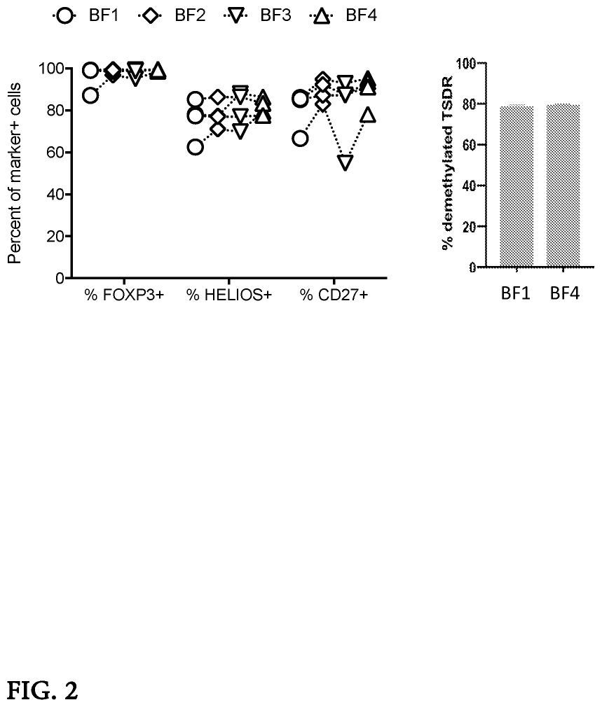

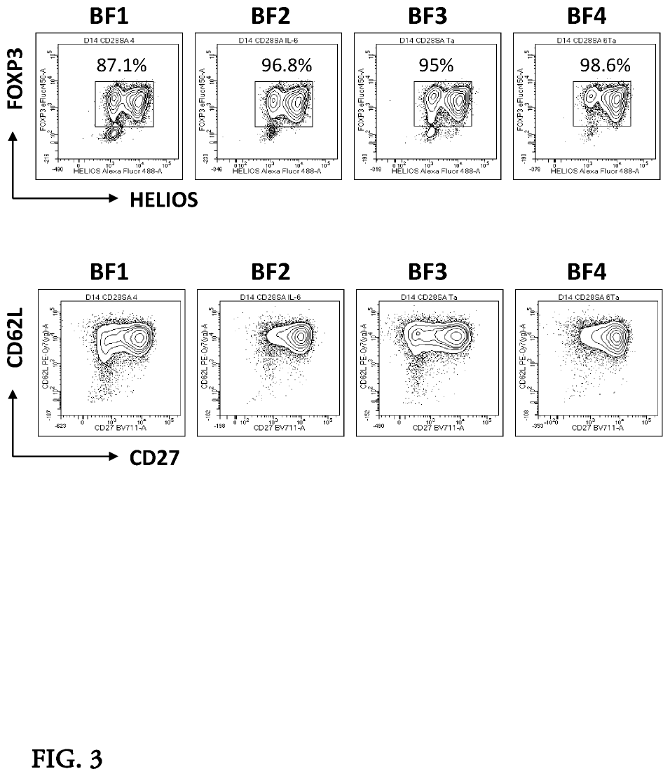

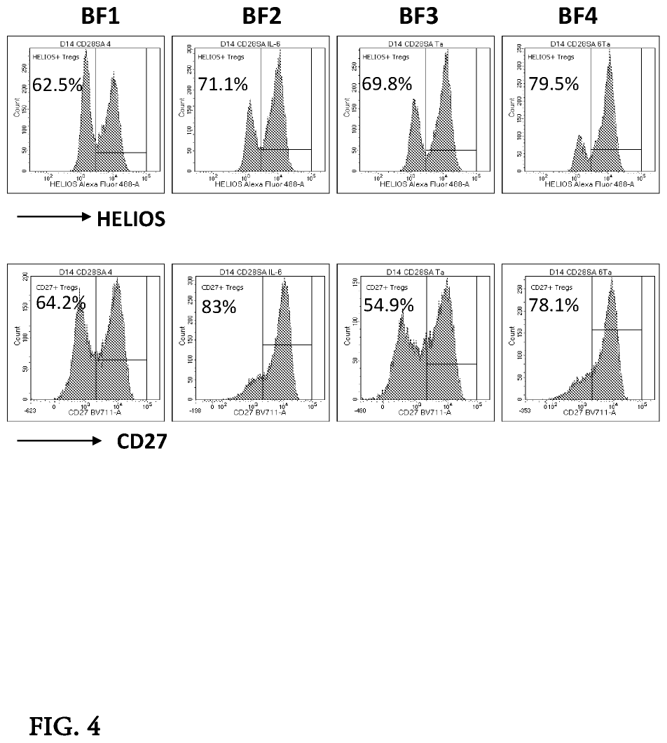

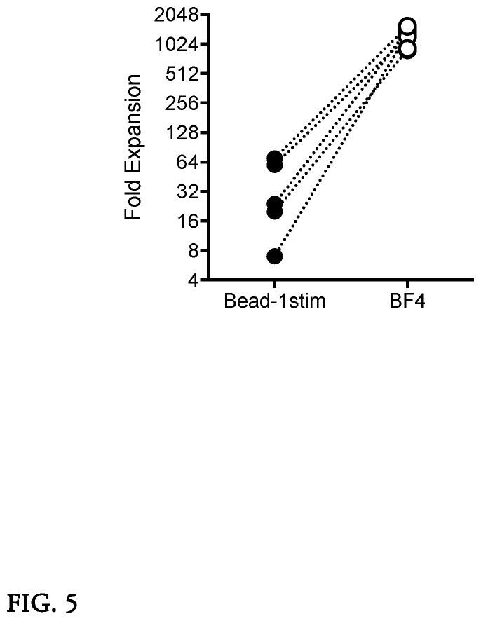

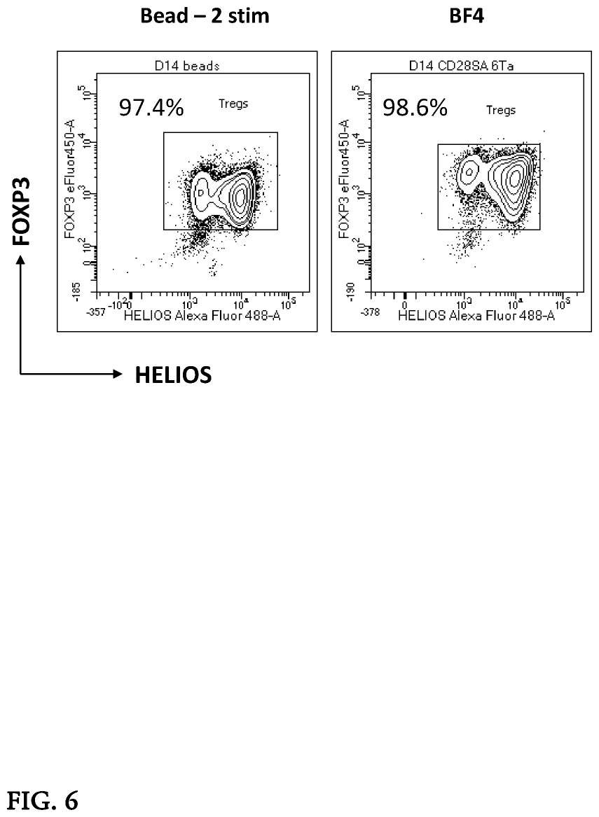

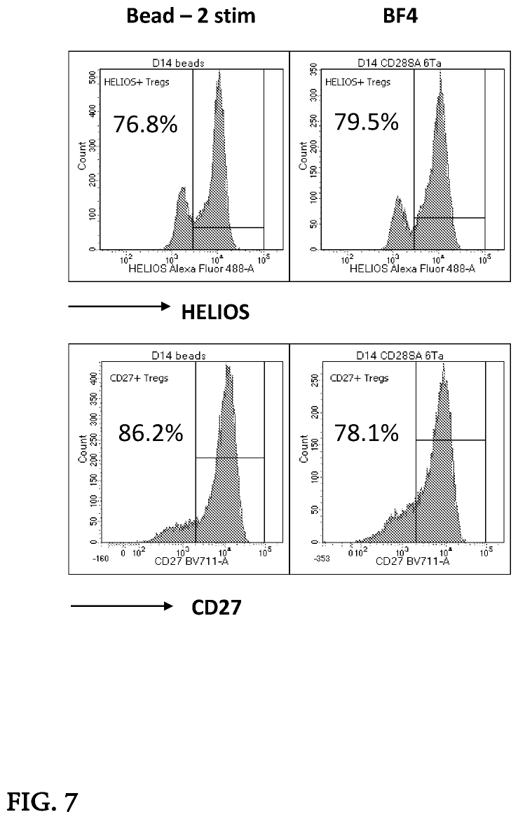

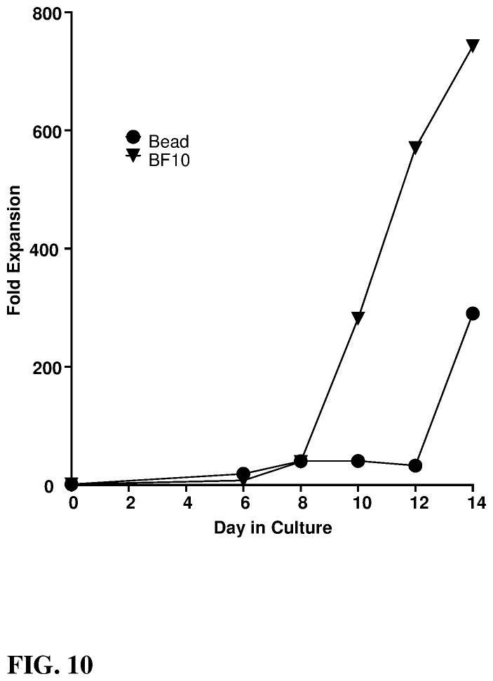

As shown in FIG. 2, ex vivo expansion of Tregs by stimulation with CD28SA Ab in the presence of proinflammatory cytokines yields a cell population that has a high level of expression of Treg lineage markers FOXP3, HELIOS, and CD27. In addition, the expanded cell population has a highly demethylated TSDR. A comparison of the phenotype of Tregs expanded ex vivo under BF1, BF2, BF3, and BF4 stimulation conditions is shown in FIG. 3 and FIG. 4. Treg expansion under the BF4 condition resulted in the production of over 1000 fold more cells than was present at the onset of stimulation (day 0), whereas the extent of Treg expansion under the standard anti-CD3/CD28 bead condition was considerably less, as shown in FIG. 5. Similarly, Treg expansion under the BF10 condition resulted in the production of far more cells than did expansion under the standard anti-CD3/CD28 bead condition, as shown in FIG. 10. A comparison of the phenotype of Tregs expanded ex vivo under the BF4 condition, and a standard anti-CD3/CD28 bead condition is shown in FIG. 6 and FIG. 7.

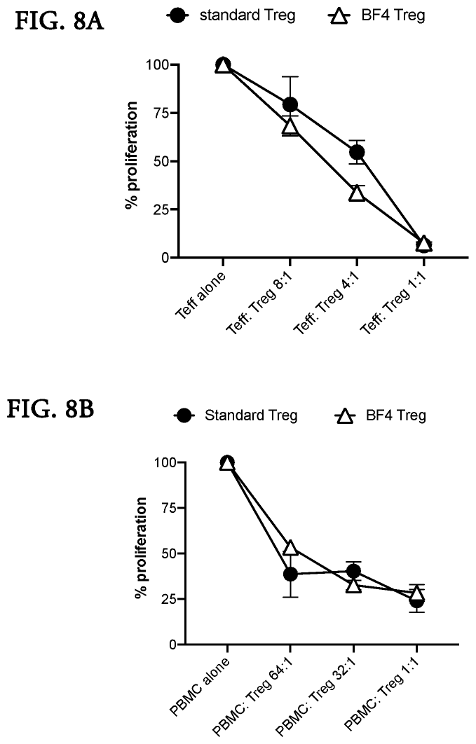

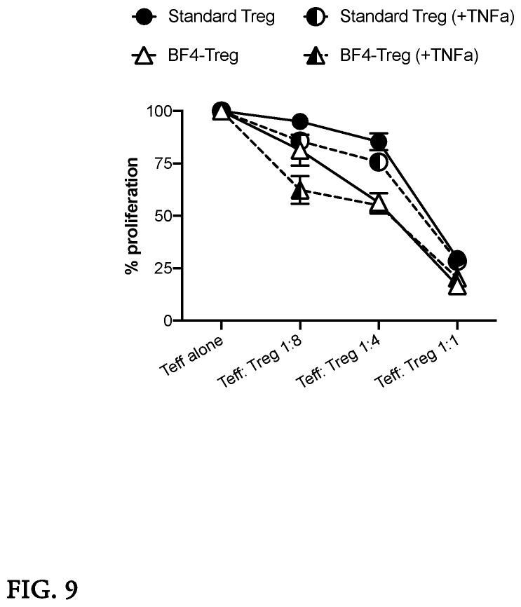

Expansion of Tregs ex vivo by stimulation with CD28SA Ab in the presence of proinflammatory cytokines under a BF4 stimulation condition yields a cell population that possesses a high suppressive capacity against pre-activated Teff and autologous PBMC as shown in FIG. 8A and FIG. 8B. Additionally, Tregs expanded ex vivo under a BF4 stimulation condition are more potent suppressors of Teff proliferation in the presence of the inflammatory cytokine TNF-alpha than are Tregs expanded ex vivo under a standard anti-CD3/CD28 bead condition as shown in FIG. 9.

Moreover, expansion of Tregs ex vivo by stimulation with CD28SA Ab in the presence of proinflammatory cytokines does not increase the frequency of Tregs that produce the proinflammatory cytokines IL-2, IL-17, IFN-gamma, and IL-4.

1. A method for the production of human regulatory T cells (Tregs), comprising:
a) isolating CD4+, CD25+, CD127−/low T cells from a lymphocyte-containing biological sample obtained from a human subject; and
b) culturing the T cells in medium comprising a CD28 superagonist (CD28SA) antibody, interleukin-2 (IL-2), and tumor necrosis factor-alpha (TNF-alpha) under conditions effective in producing human Tregs that are CD4+, FOXP3+, HELIOS+, and have a demethylated Treg-specific demethylation region (TSDR).

2. The method of claim 1, wherein step b) does not comprise use of an anti-CD3 antibody.

3. The method of claim 2, wherein step b) does not comprise use of magnetic beads or Fc receptor-expressing feeder cells to cross-link CD28 and CD3 of the isolated T cells.

4. The method of claim 3, wherein the medium further comprises one or both of a tumor necrosis factor receptor 2 agonist (TNFR2a) and interferon-gamma (IFN-gamma).

5. The method of claim 3, wherein the medium further comprises one or both of IL-6 and IL-1beta.

6. The method of claim 1, wherein the lymphocyte-containing biological sample is selected from the group consisting of whole blood, a leukapheresis product, and peripheral blood mononuclear cells (PBMC).

7. The method of claim 5, wherein the biological sample is either fresh or cryopreserved after being obtained from the human subject and subsequently thawed prior to step a).

8. The method of claim 1, wherein the CD4+, CD25+, CD127−/low T cells of step a) are isolated from the biological sample by fluorescence-activated cell sorting (FACS) or magnetic-activated cell sorting (MACS).

9. The method of claim 1, further comprising step c) harvesting the human Tregs 7-18 days after step b) commences.

10. The method of claim 9, wherein the human Tregs comprise from about 200 to about 2000 fold more cells than the CD4+, CD25+, CD127−/low T cells at the onset of step a).

11. A pharmaceutical composition comprising from 10⁷ to 10¹¹ of the human Tregs produced using the method of claim 10, and a physiologically acceptable buffer.

12. A method for treating or preventing a pathological immune response in a human subject in need thereof, the method comprising: administering to the human subject an effective amount of the pharmaceutical composition of claim 11.

13. The method of claim 12, wherein the pathological immune response is an autoimmune or autoinflammatory disease.

14. The method of claim 13, wherein the autoimmune or autoinflammatory disease is selected from the group consisting of rheumatoid arthritis, multiple sclerosis, amyotrophic lateral sclerosis, systemic lupus erythematosus, pemphigus, psoriasis, type I diabetes, celiac disease, and inflammatory bowel disease.

15. The method of claim 13, wherein the composition is effective in reducing a symptom, or is effective in inhibiting progression of the autoimmune or autoinflammatory disease.

16. The method of claim 12, wherein the pathological immune response is rejection of a hematopoietic allograft or a solid organ allograft.

17. The method of claim 16, wherein the pathological immune response is rejection of a hematopoietic allograft, and the hematopoietic allograft is a bone marrow graft or a peripheral blood stem cell graft.

18. The method of claim 16, wherein the pathological immune response is rejection of a solid organ allograft, and the solid organ allograft is selected from the group consisting of cardiac, lung, cardiac/lung, kidney, pancreas, kidney/pancreas, liver, intestine, pancreatic islet, and skin allografts.

19. The method of claim 16, wherein the composition is effective in reducing a symptom of acute and/or chronic rejection, or is effective in prolonging survival of the organ allograft.

20. The method of claim 12, wherein the pathological immune response is a graft versus host disease (GvHD).

21. The method of claim 20, wherein the composition is effective in reducing a symptom of acute and/or chronic GvHD, or is effective in inhibiting damage to skin, liver, lung, and/or gut of the host.

22. The method of claim 12, wherein the composition is effective in increasing Treg percentages over baseline in the human subject.

23. A method for inhibiting proliferation of human effector T cells (Teffs), the method comprising: contacting human CD4+, CD25−, CD127+ Teffs with the human Tregs produced using the method of claim 1 under conditions effective in inhibiting proliferation of the Teffs.

24. The method claim 1, wherein the method for production of the human Tregs is good manufacturing practice (GMP)-compliant.
